(19) United States
(12) Patent Application Publication
(10) Pub. No.: US 2004/0141912 A1
(43) Pub. Date: Jul. 22, 2004

(54) Method for targeting chemical compounds to cells and pharmaceutical compositions used therein

(76) Inventors: Ilan Ziv, Kfar Saba (IL); Anat Shirvan, Herzliya (IL); Sharon Ebner, Raanana (IL)

Correspondence Address: NATH & ASSOCIATES, 1030 15th STREET, 6TH FLOOR, WASHINGTON, DC, 20005, US

(21) Appl. No.: 10/471,725

(22) PCT Filed: Mar. 14, 2002
(86) PCT No.: PCT/IB02/00758

(30) Foreign Application Priority Data
Mar. 16, 2001 (IL) 142051
Jan. 24, 2002 (IL) 147812

(51) Int. Cl. A61K 49/04; A61K 51/00

(52) U.S. Cl. 424/1.11; 424/9.3; 424/9.4

(57) ABSTRACT

The present invention concerns a method for selectively targeting a medicinally-useful agent into cells in which a phospholipid scramblase (PLS) transport system is activated. The method comprises administering the agent, being a PLS-dependent transported compound (PDTC) to the cells, thereby causing selective transport of the agent into the cells. Examples of the cells are apoptotic cells, activated cells and injured cells. Also disclosed are pharmaceutical compositions for use by the method.

### FIELD OF THE INVENTION

The present invention relates to novel therapeutic and diagnostic methods for targeting agents to specific cell populations for diagnostic or therapeutic purposes, and to agents used in these methods. The present invention also concerns a method for screening for such agents.

### PRIOR ART

The following references are considered to be relevant for an understanding of the background of the invention:

Balasubramanian, K., et el., J. Biol. Chem., 272:31113-31117, 1997

Basse, F., et al., J. Biol. Chem., 271:17205-17210, 1996

Bevers, E M., et al., Biochim Biophys Acta 736:57-66, 1983

Bevers, E M., et al., Biochim Biophys Acta 1439:317-330, 1999

Bursch, W., et al., Trends Pharmacol. Sci., 13:245-251, 1992

Daleke, D. L., et al., Biochim. Biophys. Acta 1486:108-127, 2000

Eastman, A., Cancer Cells 2:275-280, 1990

Frasch S. C., et al., J. Biol. Chem. 275: 23065-23073, 2000

Hofstra, L., et al.,Lancet 356:209-212, 2000

Kunzelmann-Marche, C., et al., J. Biol. Chem., 276:5134-5139, 2001

Matsuno, T. et al., Transplant Proc., 28:1226-1227, 1996

Momchilova, A., et al., Arch. Biochem. Biophys. 381:295-301, 2000

Sims, P. J., et al., J. Biol. Chem. 264:17049-17057, 1989

Sulpice, J. C., et al., J. Biol. Chem. 269:6347-6354, 1994

Van-Engeland, M., et al., Cytometry, 31:1-9, 1998

Verhoven, B., et al., J. Exp. Med. 182:1597-1601, 1995

Wiedmer, T., et al., Biochim. Biophys. Acta, 1467:244-253,2000

Wyllie, A H., et al., Int Rev. Cytol 68:251-306, 1980

Zhao, J., et al., J. Biol. Chem., 273:6603-6606, 1998

Zhou, Q., et al., J. Biol. Chem. 272:18240-18244, 1997

### BACKGROUND OF THE INVENTION

Apoptosis is an intrinsic program of cell self-destruction or “suicide”, which is inherent in every eukaryotic cell. In response to a triggering stimulus, cells undergo a highly characteristic cascade of events, manifested by cell shrinkage, blebbing of cell membranes, chromatin condensation and fragmentation, culminating in conversion of the cell to clusters of membrane-bound particles (apoptotic bodies), which are thereafter engulfed by macrophages (Wyllie, A H., et al., 1980).

Apoptosis is now recognized as one of the most important biological processes, having a major role in normal tissue development and homeostasis. Moreover, derangement of apoptosis control has a role in the pathogenesis of numerous medical disorders, ranging from disorders of excessive apoptosis such as neurodegenerative disorders (e.g., Alzheimer's disease or Parkinson's disease), to disorders wherein death of defective cells is inappropriately inhibited, such as cancer (Bursch, W., et al., 1992).

One of the earliest events occurring in apoptosis, taking place around the point of cell commitment to the death process, is alterations in the normal organization of the phospholipid plasma membrane (Van-Engeland, M., et al., 1998). Normally in healthy quiescent cells, there is an asymmetric distribution of phospholipids between the outer and inner leaflets of the lipid bilayer of the plasma membrane. Aminophospholipids, mainly phosphatidylserine (PS) and phosphatidylethanolamine (PE) reside mainly in the inner leaflet of the plasma membrane, while phosphatidylcholine (PC) and sphingomyelin (SM) reside mainly in the outer leaflet. This asymmetry in phospholipid distribution is important for various cellular functions, including normal cell interaction with its environment. This phospholipid asymmetry in the plasma membrane is normally maintained, in an energy-dependent manner, by at least two main systems as follows: (1) an inward-directed pump, called the aminophospholipid translocase, which specifically and continuously moves PS and PE from the outer to the inner membrane leaflet in an ATP-dependent manner, while being inhibited by Ca²⁺; and (2) an outward directed floppase, that moves mainly PC from the inner to the outer membrane leaflet (Bevers, E M., et al., 1999).

Early in the apoptotic process, there is a rapid disruption of normal phospholipid asymmetry of the plasma membrane, leading to exposure of PS and PE on the cell surface and inward movement of PC and SM. This process promotes recruitment of tissue macrophages to engulf the apoptotic cells, and also renders the surface of the apoptotic cells highly procoagulant (Balasubramanian, K., et el., 1997). This process is universal, occurring in almost any cell undergoing apoptosis. Therefore, annexin V, a protein having a high affinity to PS, has been used for detection of the exposure of PS on the cell surface, thus serving as a diagnostic probe for apoptosis in studies both in vitro and in vivo (Hofstra, L., et al., 2000).

This disruption of phospholipid asymmetry of the plasma membrane, although very characteristic of apoptosis, also occurs in other biological situations such as cell activation or cell damage by various noxious stimuli. For example, it occurs during platelet activation, thus changing their surface into a powerful catalytic platform for the assembly of clotting factor complexes, mainly the tenase and prothrombinase complexes (Bevers, E M, et al., 1983). Similarly, it occurs in white blood cells and in tissue macrophages undergoing activation.

The factors responsible for this dramatic redistribution of plasma membrane phospholipids (“accelerated flip-flop”, “phospholipid scrambling”) are not fully elucidated yet, due to the inherent complexity of the membrane and associated protein systems (Kunzelmann-Marche, C., et al., 2001). Evidently, phospholipid scrambling results from the concerted activity of multiple factors. These include, among others, inhibition of the aminophospholipid translocase with subsequent inhibition of inward movement of PS and PE, (Verhoven, B., et al., 1995), activity of MDR1 p-glycoprotein, activity of transglutaminase, cellular polyamines, phosphatidylinositol 4,5,-biphosphate (Sulpice, J. C., et al., 1994), and derangement of proteins that normally tether the sub-membranous cytoskeleton to the membrane (Sims, P. J., et al., 1989).

Phospholipid scramblase (PLS) is a recently-discovered family of proteins that plays a role among the factors that control inter-layer movement of plasma membrane phospholipids (Zhou, Q., et al., 1997; Basse, F., et al., 1996). PLS facilitates inter-layer migration of all classes of phospholipids. However, it manifests differences in the kinetics of its activity for different phospholipid species. Said differential effect is determined by the structures of the headgroup and the phospholipid backbone, wherein lower rates of PLS-mediated movement are observed for phospholipids with a ceramide backbone (e.g., sphingomyelin), in comparison to PLS-mediated migration of glycerophospholipids (e.g., phosphatidylcholine). PLS is therefore characterized by certain structure/function specifications that determine its activity towards different substrates.

The prototypic member of this protein family is a 37 kDa protein, which is expressed in numerous cell types. The expression and activity of this protein have been found to correlate with rapid movement of PS to outer surface of the plasma membrane, such as the movement observed during cell activation or apoptosis (Zhao, J., et al., 1998; Frasch, S. C., et al., 2000). Several other members of the PLS family have also been recently cloned in humans and mice (Wiedmer, T., et al., 2000). Importantly, PLS requires Ca²⁺ for its activity. In addition, PLS activity is regulated by protein kinase Cδ (PKCδ) (Frasch, S. C., et al., 2000). Other cellular regulatory mechanisms for PLS activity are now being explored.

Sequence analysis of PLS family members reveals that each member is a single-pass transmembrane protein. The prototypic, 37 kDa PLS family member has 318 amino acid residues, and is rich in proline. It has a short 9 amino acid C-terminal extracellular sequence. Intracellularly, near the transmembrane segment, the protein comprises a putative calcium-binding loop motif of 12 amino acid residues. The intracellular segment also comprises a potential protein kinase C phosphorylation site (at Thr159), which may account for the regulation of PLS by PKCδ. In addition, the protein comprises one or more intracellular cysteinyl residues that are palmitoylated. Potentially, these thioester-linked palmitate side chains may have a role in determining the folding of the calcium-binding loop, or they could function directly in enhancing interlayer phospholipid movement. Conceivably, PLS is part of a complex, having accessory neighboring structural protein units assisting PLS in its activity. Potentially, PLS may also act as an oligomer (Daleke, D. L., et al., 2000). Therefore, either (a) member(s) of the PLS protein family; or a complex, comprising said protein(s), multimers of said protein(s), and/or associated structural units, are within the scope of definition of PLS in the context of the present invention.

To date, PLS activity has been described in relation to its effect on the intra-membrane distribution of the phospholipid constituents of the plasma membrane, i.e., phospholipid distribution between the inner and the outer membrane leaflets.

### SUMMARY OF THE INVENTION

The present invention is based on the findings, by the inventors, of activity of PLS also as a drug transporter into cells. Moreover, the inventors have characterized chemical compounds, many of which do no have phospholipid structure, that can utilize PLS for selective transmembrane transport into cells wherein PLS is activated.

Thus, in accordance with the present invention, PLS-mediated transport is used as a basis for selective targeting of molecules into cells in which the PLS transport system is activated, and as a method to distinguish between such cells and other cell types, for therapeutic and diagnostic purposes.

The PLS-dependent mechanism for rapid transport of chemical compounds into cells wherein PLS is activated is hereinafter designated “PLS-dependent transport” (PDT).

A compound delivered by the PDT is hereinafter designated a “PLS-dependent transported compound” (PDTC).

The term “PDT-activated cells” in the context of the present invention means cells manifesting higher PLS activity as compared either to cells of a different type, e.g. of a different tissue, of a different lineage, etc.; or a higher activity found in diseased cells or cells displaying certain pathological or physiological alterations, as compared to such normal (healthy, quiescent) cells of the same kind. Preferably the PDT-activated cells are selected from apoptotic cells (in particular cells in the early stages of apoptosis), cells undergoing activation (such as activated platelets, or activated inflammatory cells) and injured cells (e.g., injured red blood cells). By contrast, healthy, quiescent cells do not manifest significant PDT activity, and are therefore not considered PDT-activated cells. The present invention therefore utilizes the PDT as a method to distinguish between these cell populations, for therapeutic and diagnostic purposes.

The term “selective targeting” in the context of the present invention means binding and/or entering of a medicinally-useful agent, to PDT-activated cells at significantly higher rates than the rates of binding/entering of the agent to non-PDT-activated cells. The term medicinally-useful agent in the context of the present invention is defined as a PDTC having diagnostic or therapeutic activity. The medicinally-useful agent may comprise either one molecule, one type of molecule or a conjugate of two or more molecules whereby one or more of the molecules provides the PDTC function and one or more of the molecules provides the diagnostic or therapeutic activity. At times, the same molecule may provide both the PDTC and the diagnostic or therapeutic functions.

Thus the present invention concerns a method for selectively targeting a medicinally-useful agent into cells in which a phospholipid scramblase (PLS) transport system is activated (PDT-activated cells), wherein the medicinally-useful agent is a PLS-dependent transported compound (PDTC), the method comprising: administering the agent to the cells, thereby causing selective transport of the agent into these cells.

The present invention also provides a pharmaceutical composition comprising a pharmaceutically acceptable carrier and a medicinally-useful agent capable of being selectively targeted into PDT-activated cells, the medicinally-useful agent being a PDTC.

Also provided by the invention is the use of a medicinally-useful agent, being a PDTC, that can be transported into PDT-activated cells by PLS, for the preparation of a pharmaceutical composition for selectively targeting the agent into said cells.

The surprising finding of PDT and those PDTC transported thereby, leads the way to several utilizations. According to one aspect of the invention termed “the diagnostic aspect”, the PDT may be used to transport diagnostic compounds into PDT-activated cells, e.g., apoptotic cells (preferably cells in the early stages of apoptosis), injured cells or activated cells (such as activated platelets, inflammatory cells and white blood cells). For these diagnostic purposes, the PDTC is either the diagnostic compound itself comprising a marker moiety, or a conjugate of the diagnostic compound with a carrier moiety, said conjugate having a PDTC activity. The term “diagnostic PDTC” will be used to denote a PDTC utilized, in accordance with the invention, for diagnostic purposes.

The term “marker moiety” will be used to denote the moiety within the diagnostic PDTC that has a property that permits identification of its signal by visual or instrumental means [such as a moiety that contains a fluorescent label, radioactive label, label for X-ray imaging, label for CT scan, single photon emmision computerized tomography (SPECT), positron emission tomography (PET), magnetic resonance imaging M), etc.]. The PDTC may comprise a marker for imaging that may be a radio-isotope, e.g., ^{99m}Technetium, ¹¹¹Indium, or ¹²³I, a marker for MRI (e.g., gadolinium), a marker for computerized tomography (CT) scan, or a marker for PET (e.g., ¹⁸F). When the marker is a metal ion, the PDTC may also comprise a chelator, for the chelation of the marker [e.g., an N₂S₂ chelator, such as a chelator comprising monoamine-monoamide-bisthiol or bis-(aminoethanethiol), for chelation of technetium]. In some cases, the PDTC may also have inherent imaging characteristics, such as fluorescent properties.

A method in accordance with the diagnostic aspect of the invention involves detecting the marker moiety in the PDT-activated cells. Due to the direct correlation between PDT activation and the processes of apoptosis, cell activation or cell damage, the signal obtained from a tissue by these imaging means upon administration of a diagnostic PDTC, may be used for analysis and quantification of the number or fraction of cells within the tissue that undergo any of the above processes.

The diagnostic aspect of the invention may be utilized, for example, for detecting apoptotic cells, particularly in the early stage of apoptosis, injured cells or activated cells, particularly in their early stages of activation. Activated cells may be, for example, activated platelets; the detection of activated platelets may permit to assess the risk of blood clots or occlusion of blood vessels due to clots. Activated cells may also be activated inflammatory cells or white blood cells, in which case the diagnostic PDTC may allow a diagnosis of an inflammatory process.

In accordance with another aspect of the invention termed: “the therapeutic aspect”, the PDT may also be used to target and transport therapeutic substances into cells wherein PDT is activated [e.g., cells in apoptosis (particularly in the early stages of the death process), cells undergoing activation (particularly cells in the early stages of activation), such as activated platelets, or injured cells]. The targeting may be used for the purpose of modulating a biological activity of the cells (down or up modulation), protecting them against damage, reversing a pathological process, enhancing cell death, etc. The term “therapeutic PDTC” will be used to denote PDTCs that are selectively delivered into cells, in order to exert within the target cells an effect, such as any of those mentioned above. The term “effector moiety” will be used to denote a moiety included within the PDTC, which induces a desired effect on the cell once bound and internalized into it. Said desired effect is particularly a therapeutic effect that comprises modulating a biological activity of the cell, protecting cells against damage, reversing a pathological process or modulating a process of cell death.

For said therapeutic purposes, the PDTC is either the effector moiety itself, or a conjugate of the effector moiety with a carrier moiety, said conjugate having PDTC activity. For apoptotic cells, said therapeutic substances are preferably those modulating (either enhancing or protecting from) cell death. For activated platelets or apoptotic cells, said therapeutic substances are preferably those modulating blood coagulation [e.g., anticoagulant, fibrinolytic agents or anti-platelet agents (such as inhibitors of platelet aggregation)]. For activated inflammatory cells, said therapeutic substances are preferably anti-inflammatory drugs, or drugs capable of modulation of the immune system (i.e., immuno-modulatory drugs). Such inflammatory cells may be, for example, white blood cells or macrophages.

According to the therapeutic aspect, the method of the present invention may be utilized to improve treatment of diseases by selective targeting drugs to cells or tissues inflicted by PDT-activation, i.e., apoptotic cells; activated cells such as platelets or white blood cells; or injured cells.

At times, the PDTC or medicinally-useful agent in its entirety serves the purpose for either affecting the cell or for purposes of detection of the cell (in accordance with the therapeutic or diagnostic aspects, respectively). Alternatively however, the PDTC may comprise a defined marker moiety or an effector moiety, as the case may be, and a carrier, wherein said carrier, together with the marker moiety or the effector moiety jointly form the PDTC. The PDTC may also comprise a moiety that can act both for diagnostic and therapeutic purposes.

In accordance with one embodiment of the therapeutic aspect, the PDTC is delivered to cells for an essentially immediate effect. In accordance with another embodiment of the therapeutic aspect, the PDTC is delivered to cells in order to create a depot of a therapeutic substance in the cells in which the PDT is active (for example, in cells in the early stage of apoptosis, activated cells such as platelets or inflammatory cells, or injured cells), for the purpose of subsequent gradual release of the effector moiety within the cells. Optionally, the release may also be a PDT-dependent process, in which the PDT thus acting not only to target drugs to the cells, but also subsequently to allow their delayed sustained release from the intercellular storage. An example of a therapeutic PDTC in accordance with this embodiment, is a molecule that can modulate blood coagulation. A PDTC that can modulate blood coagulation may be selected from from an anticoagulant, an anti-platelet agents and a fibryinolytic agent, being targeted in accordance with the invention into activated platelets or apoptotic endothelial cells within a blood clot, with consequent accumulation therein. In this way, the PDTC may exert an inhibitory effect on the thrombotic process in situ, namely at the site of thrombus formation. Other examples of therapeutic PDTCs in accordance to this embodiment is are anti-inflammatory drugs or immuno-modulatory drugs, thus being targeted to foci of inflammation, or modulators (inhibitors or enhancers) of apoptosis, being targeted to foci of apoptosis.

Finally, the above finding may lead the way to a method for screening and finding novel compounds, to be targeted to apoptotic, injured or activated cells. The identification of such compounds may be desirable for either diagnostic or therapeutic purposes. Such a screening method may be based on screening of compounds for their ability to serve as PDTCs.

A PDTC according to the invention has the following characteristics:

(a). its transport into cells is augmented upon activation of PLS of said cells, said activation resulting, for example, from elevation of intracellular Ca²⁺ levels; and

(b). its transport into cells, wherein PLS is activated, is reduced upon inhibition of PLS of said cells, e.g., by removal of Ca²⁺, or by inhibition of PKCδ (e.g., by an inhibitor such as rottlerin), under appropriate conditions (Frasch, S. C., et al., 2000).

Preferably, a PDTC according to the invention also has one or both of the following additional characteristics:

(c). it has an amphipathic structure, i.e., having both hydrophilic and hydrophobic moieties; preferably, the hydrophobic moiety is selected from aromatic groups(s), aliphatic group(s), hydrophobic metal chelate(s) comprising metal atom(s), and combinations thereof; preferably, the hydrophilic moiety is charged at physiological conditions; advantageously, said hydrophilic moiety is in zwitterion form, or is a negatively-charged group at physiological conditions. In another preferred embodiment, said hydrophobic metal chelate(s) comprise(s) technetium.

(d). it has an affinity to a phospholipid membrane leaflet; said affinity may be characterized, for example, by an octanol: water partition coefficient of at least 10:1.

Where the PDTC includes two moieties, namely a marker moiety or an effector moiety on the one hand, and a carrier moiety on the other hand, the link between the two may be designed to be cleaved within the cells, whereby the marker moiety or the effector moiety is released. Preferably, said cleavage is performed by intracellular enzymes.

In the case that the PDTC comprises a drug to be targeted to PDT-activated cells which are apoptotic cells or injured cells, the drug is preferably a modulator of cell death. Advantageously, said drug is an inhibitor or an enhancer of apoptosis.

For example, inhibition of apoptosis may be desired for the treatment of either chronic medical disorders (e.g., neuro-degenerative disorders), or acute medical disorders such as myocardial infarction or cerebrovascular stroke. In such cases, the PDTC may be, for example, a caspase inhibitor, a modulator of Bcl-2, an antioxidant or another cyto-protective compound.

Enhancement of cell death may be required, for example, in the treatment of cancer, wherein elimination of defective, hazardous cells should be enhanced. One embodiment of the invention is a PDTC, comprising an anti-cancer, cytotoxic agent, capable of inducing or enhancing apoptosis in tumor tissue.

In the case that the PDT-activated cells are activated platelets, the PDTC to be targeted to the activated platelets by the method of the present invention, can be an inhibitor of platelet activity (e.g., an inhibitor of platelet aggregation), an inhibitor of blood coagulation, or a fibrinolytic agent.

In the case that the PDT-activated cells are activated white blood cells (WBC) such as lymphocytes or macrophages, the drug to be targeted, by the method of the present invention, may be an anti-inflammatory drug, an immuno-modulatory drug or a modulator of macrophage activity.

Optionally, the PDTC of the present invention may also comprise a moiety, capable of enhancing the trapping of the PDTC within the cell after its entry (hereinafter designated “trapping enhancing moiety” (TEM). This moiety aims at helping reducing efflux of the drug from the cell after its entry. Preferably, said TEM comprises a group that dissociates from the PDTC, selectively after entry into the cell, so that the product of said dissociation is maintained inside the cell, for example, due to its lipid solubility or electrostatic properties. Such dissociation may be due to cleavage by intracellular enzymes (e.g., esterases).

The effector moiety of a therapeutic PDTC may also be a drug known for its therapeutic activity, conjugated to a carrier moiety, as described above. Said drug can thus be selectively targeted only to cells, tissues or organs undergoing PDT activation. Once the drug reaches the target, it can exert its therapeutic activity there. The drug may exert the therapeutic activity, either when still being a part of the conjugate, or after its disconnection therefrom (for example, by cleavage, destruction, etc., performed by activity of natural enzymes or by any other known mechanism). In the case that said PDTC comprises a TEM, said TEM may act to augment the accumulation of the drug within the cells.

The effector moiety should be chosen in accordance with the specific disease, for which the composition is intended. In the case that the disease is manifested by inappropriate and excessive apoptosis, such as neurodegenerative disorders, myelodysplasic syndrome, AIDS, various ischemic or toxic insults, or graft loss following organ transplantation, the drug should preferably be capable of inhibiting apoptosis. Such drug may be, among others, a caspase inhibitor, an anti-oxidant or another apoptosis-inhibitory drug.

For the purpose of enhancing the efficacy of anticancer protocols, the PDTC may comprise an anticancer drug as an effector moiety. The enhancement of the anti-cancer protocol is achieved by either: (1) targeting of said anticancer drug to the cancer tissue; abnormally excessive apoptosis often occurs within a tumor, parallel to the abnormal tissue proliferation, and said apoptosis is often positively correlated with the level of tumor aggressiveness; or (2) use of two waves of apoptosis: the first wave is achieved by using a standard chemotherapeutic or radiotherapeutic agent, aimed at initiating/augmenting the apoptotic process within the tumor; followed by a second wave of apoptosis, induced by the PDTC comprising the anticancer drug, upon targeting of said PDTC to the foci of apoptosis produced by the first wave. By these embodiments of the method of the invention, the PDTC, comprising the anticancer drug, is targeted to the apoptosis-inflicted regions within the tumor, thus enhancing the local tumor-killing process.

In the case that the disease is associated with platelet activation, the effector moiety should preferably be capable of inhibiting platelet activity and/or blood clotting. Such an effector moiety may be an anti-platelet drug, an anticoagulant or a fibrinolytic agent. Such diseases may be, for example, atherosclerosis, arterial or venous thrombosis, thrombo-embolism, myocardial infarction, cerebral stroke, deep vein thrombosis, disseminated intravascular coagulation (DIC), thrombotic thrombocytopenic purpura (TTP), sickle cell diseases, thalassemia, antiphospholipid antibody syndrome, systemic lupus erythematosus, etc.

In the case that the disease is associated with excessive activation of white blood cells (WBC) or macrophages, the effector moiety should preferably be an anti-inflammatory drug or an immuno-modulatory drug. Such disorders include, among others, autoimmune disorders such as systemic lupus erythematosus (SLE), rheumatoid arthritis, scleroderma, or other types of connective tissue disorders; thyroiditis; dermatological disorders such as pemphigus or erythema nodosa; autoimmune hematological disorders; autoimmune neurological disorders such as myasthenia gravis; vasculitis; inflammatory bowel disorders such as ulcerative colitis, etc.

The present invention also concerns a method for screening for and identifying a PDTC, thus finding novel compounds to be targeted selectively to apoptotic, injured or activated cells. A PDTC according to the invention may be selected based on the dependency of its transport into cells on the level of the cells' PLS activity. Therefore, the ability to modulate transport of PDTCs into cells by providing conditions that modulate PLS activity of said cells, provides a means for such a selection of a PDTC. Said transport can be controlled, for example, by manipulations of cellular PKCδ activity and/or manipulation of ambient Ca²⁺ levels (under physiological conditions such as temperature and pH), e.g., a compound manifesting relatively rapid transport into cells in the presence of Ca²⁺ and PKCδ activity, while manifesting reduced transport in the absence of at least one of these conditions, is a likely PDTC candidate.

For example, compounds may be screened by their incubation with cells having an active PDT, and monitoring of the transport of said compounds into said cells. The compounds selected by such screening may then be further screened for such transport under conditions that inhibit PDT, such as depletion of Ca²⁺.

In practice, the assay for identifying likely candidates for serving as PDTCs may be carried out by placing known concentrations of various candidate compounds outside the cell, manipulating PKCδ activity, and/or manipulating ambient (intracellular and extracellular) Ca²⁺ levels, and subsequently measuring compound levels within the cell. Inhibition of PKCδ activity can be achieved by administration of an inhibitor of this enzyme, such as rottlerin. Activation of PDT by elevation of Ca²⁺ levels may be achieved, for example, by raising Ca²⁺ concentrations in the medium wherein cells are incubated and concurrent administration of a calcium ionophore such as A-23187. Inhibition of PDT by reduction of Ca²⁺ concentrations may be achieved by high-affinity extra- and intracellular Ca²⁺ chelators, such as EGTA [ethylene glycol-bis(beta-aminoethyl ether)-N,N,N′,N′-tetracetic acid] and BAPTA, [1,2-bis(2-aminophenoxy) ethane-N,N,N′,N′ tetracetic acid], respectively (Momchilova, A., et al., 2000). Measurements of intracellular drug levels can be performed by standard procedures, known to those of art.

For the purposes of the present invention, the ratio between the intracellular concentration achieved upon activation of PDT, and the intracellular concentration achieved upon inhibition of PDT should preferably be >2.

In a specific embodiment, the assay for identifying likely candidates for serving as PDTCs comprises:

(a) providing a cellular system, wherein PLS and PKCδ are expressed and functional;

(b) providing conditions suitable for PDT activation;

(c) placing known concentrations of the candidate compound outside the cellular system, and measuring the concentration of the compound, achieved in the cell after a time period;

(d) repeating stages (a) and (c), but replacing step (b) by providing conditions under which PDT activity is inhibited;

Compounds having at least two-fold higher intracellular concentrations in (c) as compared to the concentration measured in (d) are likely candidates for serving as PDTC. Preferably the time period should be 3-5 minutes.

### DESCRIPTION OF SPECIFIC EMBODIMENTS

A specific group of compounds that may be used as PDTCs in the method of the present invention are those described in the Applicants' co-pending PCT Application No. PCT IB/01/02282 and having the formula Cₑ wherein e is selected among 1, 2 and 3 and C is a group having the formula (I):

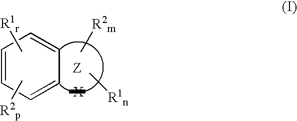

including pharmaceutically acceptable salts and hydrates of the structure of formula (I), wherein said C groups may each be the same or different and;

Z represents a ring system formed of cycloalkyl, cycloalkenyl, heterocyclyl, aryl or heteroaryl groups or combinations of such groups, said ring system consisting of 5-10 atoms;

X represents CH, CH₂, N, NH, O or S;

n, m, r and p are each independently 0 or 1; wherein n+r=1; m+p=1;

R¹ groups may each be the same or different and are independently selected from the group consisting of A, and L-A, wherein L groups may each be the same or different and are independently selected from the group consisting of D, U, U-D, D-U, D-U—O, O—U-D, D-U—NH, NH—U-D, D-U-D, and U-D-U;

U stands for a hydrogen or is selected from optionally substituted C₁-C₁₀ Is alkylene, C₂-C₁₀ alkenylene, C₃-C₁₀ branched alkylene, C₃-C₁₀ branched alkenylene, C₃-C₆ cycloalkylene, cycloalkenylene, aryl, heterocycloalkylene, heterocycloalkenylene, heteroaryl, and any combinations of said groups;

D is selected from the group consisting of O, S, SO, SO₂, SO₂NH, NHSO₂, NH, PO, PO₂, POOH, PO(NH)₂, NHPOOH, CO, C(O)O, NHCO, CONH, SO₂NHCHCOOH, SO₂NHCO or the corresponding meaning from the above list when D is a bivalent radical;

A groups may each be the same or different and are charged moieties at pH of about 7 when e is 1; or when e is 2 or 3, A groups are independently selected from polar uncharged moieties and charged moieties at pH of about 7, said charged moieties being either positively-charged, negatively-charged or in zwitterion form;

R² is WR³_{b}, where W is null or is selected from the group consisting of secondary or tertiary amine, oxygen, sulfur and D, wherein D is as defined above;

b is 1, 2 or 3,

R³ is selected from hydrogen, C₁-C₆ alkyl, C₂-C₆ alkenyl, C₃-C₈ branched alkyl and C₃-C₈ branched alkenyl; and when b is 2 or 3, R³ moieties may be either the same or different, and

when e is 2 or 3, the C groups are linked to each other either directly or through an L moiety.

The PDTC of formula Cₑ may comprise, or be attached through an L group to a marker moiety or to an effector moiety as defined above, wherein L group is as defined above.

Specific examples of PDTCs are the following:

Compounds of the formula (V):

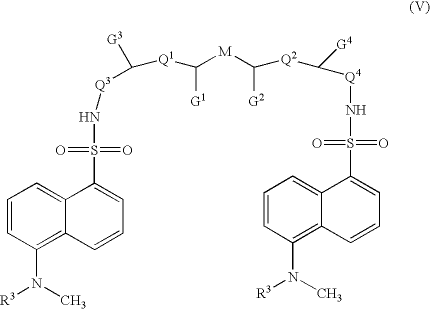

including pharmaceutically acceptable salts and hydrates thereof, wherein G¹, G², G³ and G⁴ groups may be the same or different and are selected independently among hydrogen, COOH, C(O)NH₂, NH₂, and —N⁺(CH₃)₃; M is selected among null, C(O)NH, NH, O, S, S—S, CH₂, (CH₂)₂—, NH(CH₂)₂H, N(CH₂)ₖCH(COOH) and N⁺(CH₃)(CH₂)ₖCH(COOH); Q¹, Q², Q³ and Q⁴ groups may be the same or different and are selected among null or (CH₂)ₖ, k being an integer of 1-6, and R³ represents hydrogen or a C₁-C₆ alkyl;

Compounds of the formula (VII):

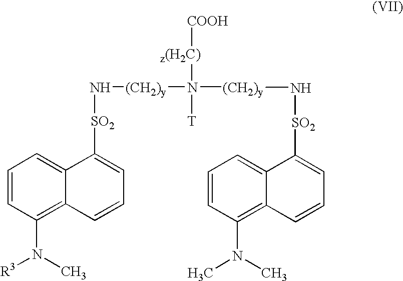

including pharmaceutically acceptable salts and hydrates thereof, wherein R³ substituents may be the same or different and are independently selected from the group consisting of hydrogen, C₁-C₆ alkyl, C₂-C₆ alkenyl, C₃-C₈ branched alkyl, and C₃-C₈ branched alkenyl, y stands for an integer of 2-6, z stands for an integer of 1-6, and T is selected among null, hydrogen and methyl;

Compound of the formula (VIII):

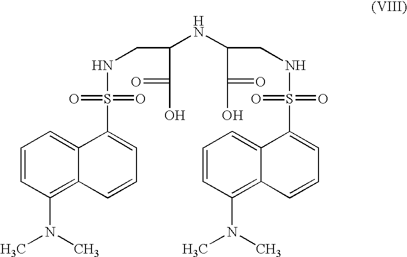

including pharmaceutically acceptable salts and hydrates thereof;

Compounds of the formula (IX):

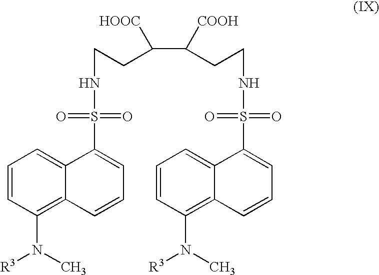

including pharmaceutically acceptable salts and hydrates thereof, wherein R³ represents hydrogen or methyl; in the case that R³ is methyl, the compound is designated NST830.

Compound of the formula (X), also designated hereinafter DDL:

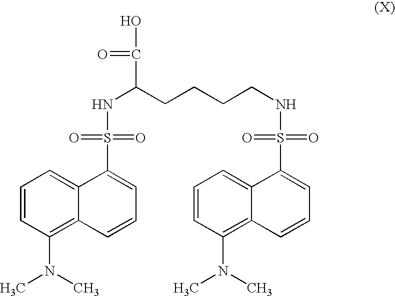

including pharmaceutically acceptable salts and hydrates thereof; and

Compound of the formula (XI), also designated hereinafter DDC:

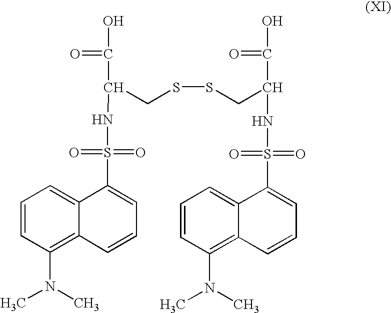

including pharmaceutically acceptable salts and hydrates thereof.

Diseases Characterized by PDT-Activated Cells which are Apoptotic:

Diseases which are characterized by occurrence of excessive apoptosis, such as neuro-degenerative disorders (e.g., Parkinson's disease, Alzheimer's disease, Huntington chorea), AIDS, myelodysplastic syndromes, ischemic (e.g., myocardial infarction, cerebrovascular stroke) or toxic insults, graft cell loss following organ transplantation. Atherosclerotic plaques, especially vulnerable/unstable plaques are also characterized by apoptosis of endothelial cells, smooth muscle cells and macrophages. Tumors, and especially highly malignant/aggressive tumors, are also often characterized, in addition to the excessive tissue proliferation, by an increased number of apoptotic cells.

Diseases Characterized by PDT-Activated Cells due to Cell Activation (e.g., Activated Platelets or Activated White Blood Cells):

1. Diseases manifested by excessive blood clotting, wherein PDT activation occurs during platelet activation, and/or during activation of or damage to other cellular elements (e.g., endothelial cells). These diseases include, among others, arterial or venous thrombosis or thrombo-embolism (myocardial infarction, cerebral stroke, deep vein thrombosis, disseminated intravascular coagulation (DIC), thrombotic thrombocytopenic purpura (TTP), sickle cell diseases, thalassemia, antiphospholipid antibody syndrome, systemic lupus erythematosus; unstable atherosclerotic plaque.

2. Inflammatory or immune-mediated diseases, associated with activation of WBC or tissue macrophages; these diseases include, among others, autoimmune disorders such as stystemic lupus erythematosus (SLE), rheumatoid arthritis, scleroderma, or other types of connective tissue disorders; thyroiditis; dermatological disorders such as pemphigus or erythema nodosa; autoimmune hematological disorders; autoimmune neurological disorders such as myasthenia gravis; vasculitis; inflammatory bowel disorders such as ulcerative colitis, organ transplant rejection, etc.

The detection of these pathological conditions, disorders or diseases may be an aim by itself, simply for the diagnosis of the presence of a disease condition in a specific individual.

Said detection may also be carried out in a person already known to have the disease, for the purpose of evaluating disease severity, characterization of disease course, or in order to monitor response to various therapeutic modalities. An example for such monitoring is evaluation of response to anticancer therapy. Most anti-tumor drugs exert their effect by induction of apoptosis (Eastman, A., 1990). Therefore, a PDTC can be useful for detection of apoptosis induced in a tumor by an anti-cancer therapy. Such imaging of apoptosis, via detection of PDT activation within the tumor, should allow optimization of anticancer treatment, by better and direct evaluation of the potency of the treatment modality in inducing death of tumor cells.

Moreover, said detection may be used to monitor adverse effects of anti-cancer treatments. A large part of such adverse effects is due to induction of untoward apoptosis by the anti-cancer treatment, in normal yet sensitive cells such as those of the gastrointestinal epithelium or the bone marrow hematopoietic system. Detection of such apoptosis using a PDTC may allow early diagnosis of such untoward tissue damage and better optimization of the treatment protocol.

In addition, said detection may aim at characterization of the intrinsic apoptoic load (i.e., the fraction of cells undergoing apoptosis) within a tumor; said load is known to be often correlated with the level of aggressiveness of the tumor. Detection of said apoptotic load may also assist in detection of metastases.

Similarly, the PDTCs may be useful in monitoring graft survival after organ transplantation, since apoptosis, potentially detectable by the method of the invention, plays a major role in cell loss during graft rejection (Matsuno, T. et al., 1996).

In addition, said detection may aim at monitoring response to cyto-protective treatments, aimed at inhibiting cell death, and thus aid in screening and development of drugs capable of such activity for various diseases (for example those recited above), by enabling a measure of evaluation of cell death.

The detection may also take place for basic research purposes, in the study of apoptosis, cell activation or cell injury, in tissue culture or animal studies, of physiological or pathological conditions associated with PDT activation, both in vitro and in vivo.

### BRIEF DESCRIPTION OF THE DRAWINGS

The following examples demonstrate the requirement for PLS activation for the uptake of the compounds DDL, DDC and NST830 into cells, wherein these compounds act as PDTCs. In these examples, PLS activation is induced in the cells by triggering of apoptosis or by cell activation. PLS inhibition in said PLS-activated cells is exemplified by deprivation of Ca2+, by inhibition of PKCδ, or by inhibition of the apoptotic cellular machinery. The inherent fluorescent properties of DDL, DDC and NST830 are utilized for visualization by either fluorescent microscopy or by flow cytometric analysis (FACS) of binding of these compounds to said cells. The assessment is focused on cells in the early stages of either apoptosis or activation, wherein cell membrane integrity is still maintained, thus excluding non-selective bulk entry of compounds into the cells via pores in the membrane. It can readily be appreciated by the artisan, that these specific embodiments are an illustration of the much broader scope of the invention as described and defined hereon.

In the accompanying figures:

FIG. 1 shows fluorescent microscopy of binding of DDL to apoptotic cultured Jurkat cells. Transport of the compound into the apoptotic cells was directly correlated with the level of PLS-activity: transport into the PLS-activated apoptotic cells was inhibited by PLS-inhibition through Ca2+ deprivation (b versus a), and promoted upon PLS-reactivation by restoration of Ca2+ (c).

FIG. 2 shows flow-cytometric analysis of binding of DDL to apoptotic cells in an aged culture of HeLa cells. DDL-binding was correlated with the level of PLS-activity: transport into the PLS-activated apoptotic cells was inhibited through PLS-inhibition by Ca²⁺-deprivation, and promoted upon PLS-reactivation by restoration of Ca²⁺. The figure shows quantification of the signal via calculation of the geomean of the fluorescence intensity.

FIG. 3 shows flow cytometric (FACS) analysis of binding of DDC to cultured Jurkat cells, induced to undergo apoptosis by anti-Fas antibody. UV-axis denotes fluorescence intensity of DDC; FL₂-axis denotes fluorescence intensity of propidium iodide (PI), a marker of membrane disintegration during late phases of cell death. Activation of PLS due to the apoptotic process resulted in the emergence of a large population of cells in the early stages of apoptosis, that manifested marked uptake of DDC, yet maintained membrane integrity (i.e., did not bind PI; B versus A, right lower quadrant). Inhibition of PLS in said apoptotic cells by addition of a low-dose (1 μM) of rottlerin, an inhibitor of PKCδ, markedly inhibited uptake of DDC by these cells in the early stages of apoptosis (right-lower quadrant; C and D)].

FIG. 4 shows flow cytometric (FACS) analysis of uptake of DDC by cultured Jurkat cells induced to undergo apoptosis by anti-Fas antibody. UV-axis denotes fluorescence intensity of DDC; FL₂-axis denotes fluorescence intensity of propidium iodide (PI), a marker of membrane disintegration during late phases of cell death.

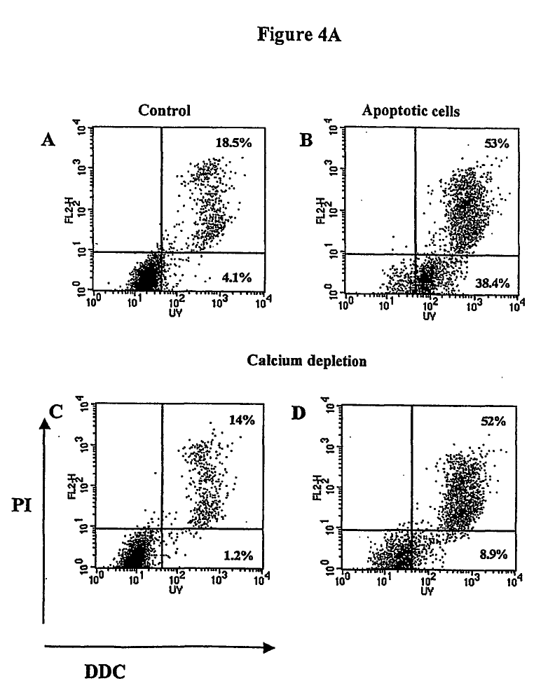

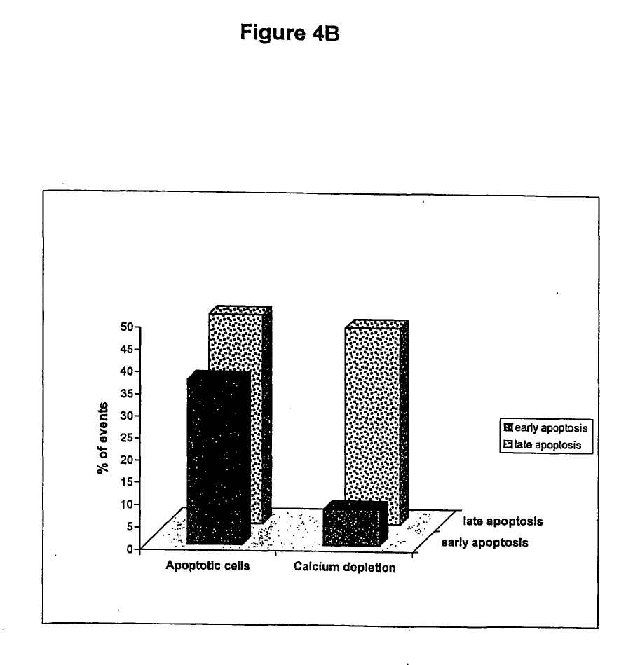

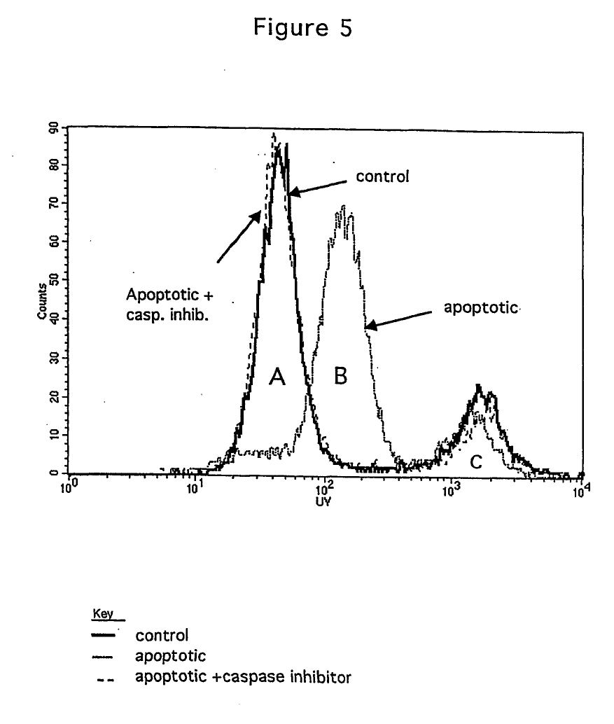

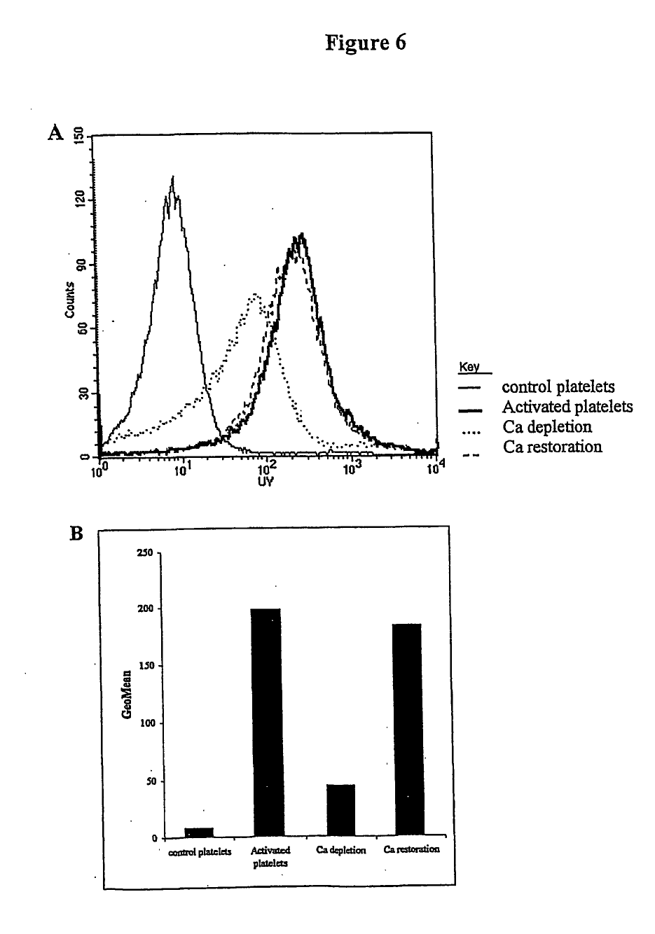

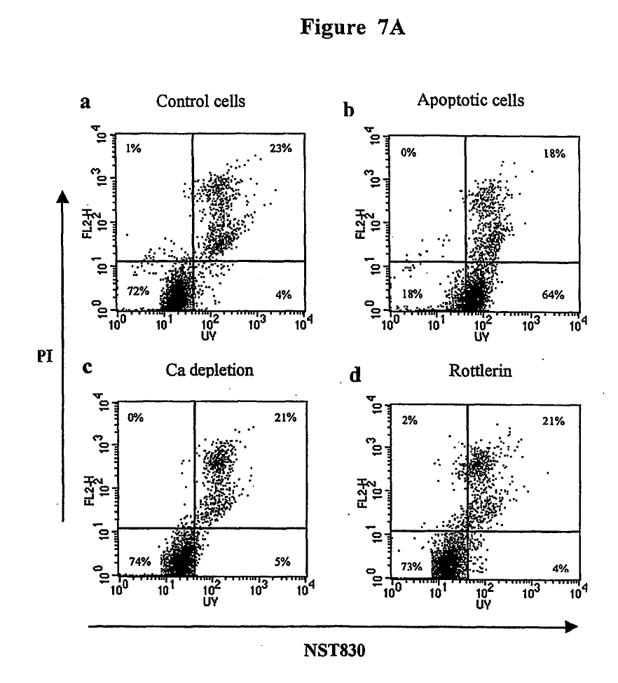

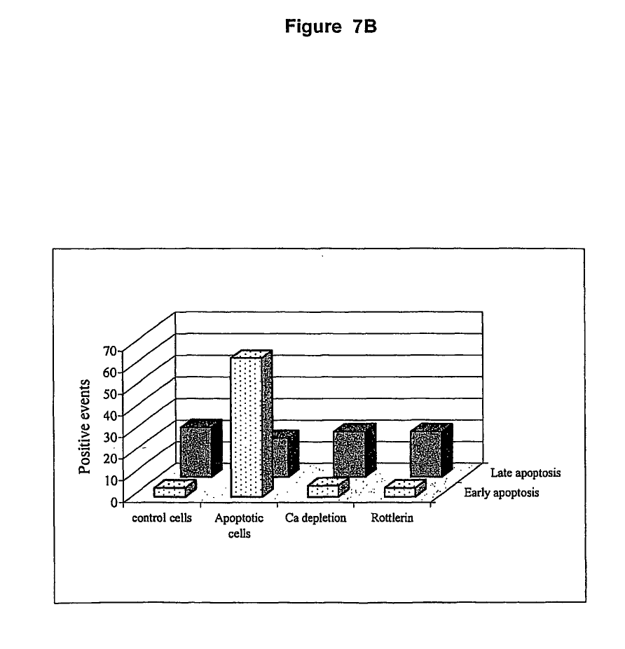

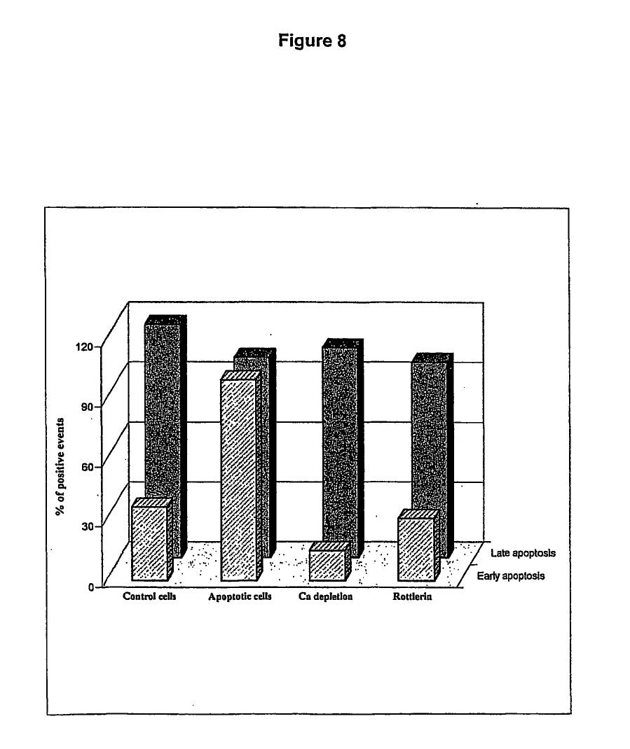

(FIG. 4A) is FACS dot plot showing the effect of Ca²⁺ depletion on binding of DDC to cells in early apoptosis. In the presence of Ca²⁺, DDC manifested marked binding to the cells (right lower quadrant) (B versus A). However, inhibition of PLS performed by deprivation of Ca²⁺ (D versus B) was associated with a marked inhibition of DDC uptake.

(FIG. 4B). Quantification of uptake of DDC by cells in the early stages of apoptosis (dark columns). Uptake was blocked by inhibition of PLS through Ca²⁺ deprivation. As an internal standard, binding of DDC to cells in the late stages of the apoptotic process is shown by the dotted columns. Upon losing cell membrane integrity in these cells, molecules can freely ingress into the cells independently of PLS, and therefore no effect of PLS-inhibition on DDC uptake by the cells in late apoptosis was observed.

FIG. 5: Shows cellular uptake of DDC, upon inactivation of PLS via blocking of the apoptotic cellular machinery by a caspase inhibitor. Cultured Jurkat cells were induced to undergo apoptosis by anti-Fas antibody. The caspase inhibitor Z-VAD-fmk was used to inhibit the apoptotic cellular machinery in these cells. The UV-axis reflects intensity of DDC binding, while the counts axis reflects the number of events. As shown, inactivation of PLS through inhibition of the apoptotic machinery prevented uptake of DDC into cells in early apoptosis.

FIG. 6 shows a flow-cytometric SACS) analysis of control and activated platelets incubated with DDC, and the effects of alterations of Ca²⁺ levels on binding of the compound. Uptake was inhibited by deprivation of Ca²⁺, and promoted by restoration of Ca²⁺. (A) FACS histogram (UV-axis denotes fluorescence intensity of DDC, while the counts axis denotes the percentage of events). (B): Quantification of DDC-binding, expressed as the geomean of the fluorescence intensity.

FIG. 7 shows a representative flow-cytometric (FACS) analysis of uptake of NST830 by Jurkat cells, induced to undergo apoptosis by treatment with anti-Fas antibody, as compared to uptake by control cells, and the inhibitory effect of PLS inhibition on said uptake.

(FIG. 7A) is a FACS dot plot, showing that activation of apoptosis leads to a marked uptake of NST830 by cells in the early stages of apoptosis, as compared to control cells (a vs. b). Inhibition of PLS activity by either deprivation of Ca²⁺ (c), or by treatment with rottlerin (d), leads to a dramatic inhibition of NST830 uptake by the cells in early apoptosis (right lower quadrant). UV-axis denotes fluorescence intensity of NST830; FL₂-axis denotes fluorescence intensity of propidium iodide (PI).

(FIG. 7B) presents quantification of the uptake of NST830 into cells in early (light, dotted columns) versus late (dark columns) apoptosis. Y-axis denotes the percent of positive events. Uptake of NST830 is found to be directly correlated with the level of activity of the PLS system: while only residual uptake is observed in the control cells wherein PLS is non-active, marked uptake is observed upon PLS-activation by the apoptotic process. PLA inhibition via either Ca²⁺-depletion or rottlerin treatment reduces NST830-uptake by cells in early apoptosis. In all cases, binding of NST830 to cells in late apoptosis, wherein uptake is not a PLS-dependent process but rather an ingress through pores in the disintegrated membrane, is not affected by PLS inhibition.

FIG. 8 shows the uptake of DDC into Jurkat cells induced to undergo apoptosis by treatment with staurosporine, and blocking of DDC-uptake by PLS inhibition via either Ca²⁺-depletion or by rottlerin treatment. Uptake into early (striped columns) versus late (dark columns) apoptotic cells is presented. The y-axis denotes the percentage of positive events for DDC staining under the different treatments. The number of events of the apoptotic culture is regarded as 100%. While PLS-activation due to induction of apoptosis leads to a marked uptake into cells in early apoptosis, DDC-uptake is markedly reduced following PLS-inhibition by either Ca²⁺-depletion or rottlerin treatment. Importantly, cells in late apoptosis, wherein uptake is not PLS-mediated, do not exhibit alterations in DDC-binding in response to inhibition of PLS.

### DETAILED EXPLANATION OF FIGURES AND EXPERIMENTAL PROCEDURES

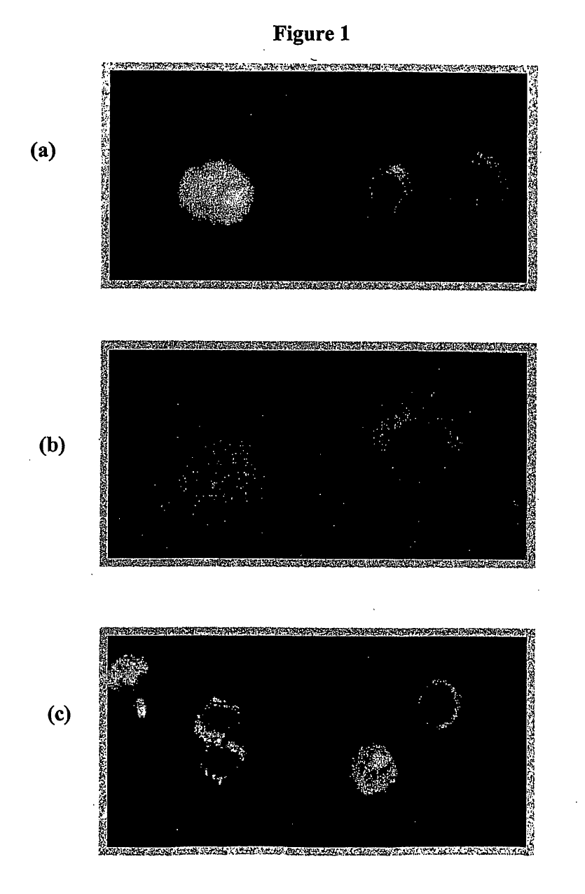

FIG. 1: Jurkat cells were treated with anti-Fas antibody (0.1 μg/ml) for three hours. As a result, a marked percentage of the cells became apoptotic (i.e., PLS-activated cells). For subsequent inhibition of PLS, culture was then treated for 30 minutes with EGTA (1 mM) and BAPTA (20 μM), (i.e., PLS-inhibited culture). Both cultures were treated with DDL (1 μM, 2 minutes) and visualized by a fluorescent microscope (Olympus):

(a) PLS-activated culture: Some of the cells manifest DDL binding, reflecting the apoptotic process occurring in these cells. DDL is shown to internalize to the apoptotic cells, staining the cytoplasm but not the nucleus.

(b) PLS-inhibited culture: a marked reduction in the intensity of DDL-binding is observed.

(c) Reactivation of inhibited-PLS: Re-activation of PLS by administration of Ca²⁺ (1 mM) and calcium ionophore A23187 (2 μM) to the same culture as in (b), resulted in re-acquisition of a strong DDL uptake. Magnification is X400.

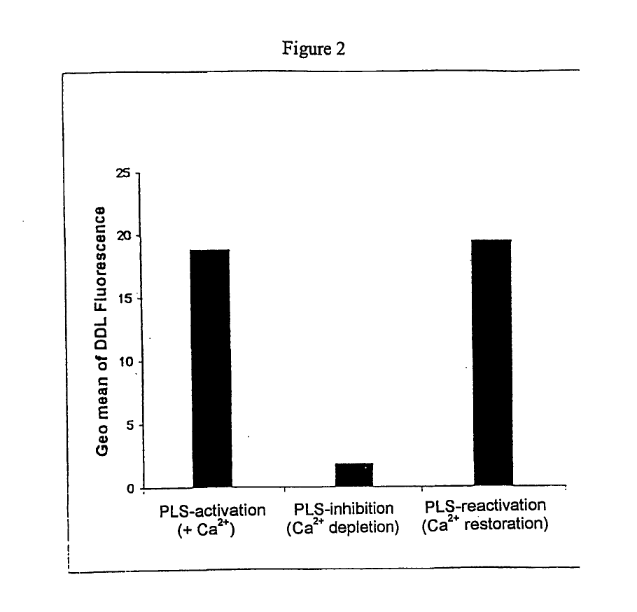

FIG. 2: HeLa cells were allowed to age by incubating the culture for 96 hours without exchange of the growth medium. As a result, a marked percentage of the cells became apoptotic. Cells were then incubated with DDL (1 μM, 2 minutes) under three different conditions: PLS-activation; PLS-inhibition; and reactivation of inhibited-PLS as defined above. Shown is quantitative analysis of fluorescence intensity (geomean) values.

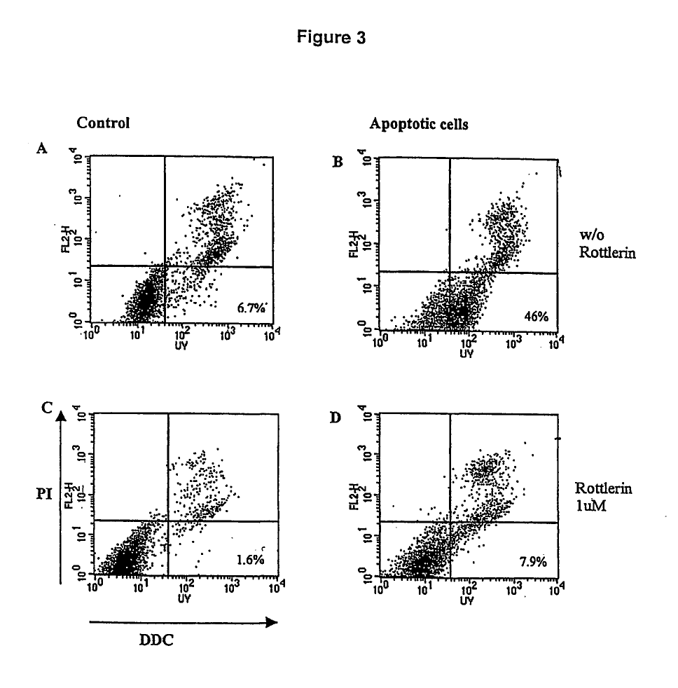

FIG. 3: Cultured Jurkat cells (A) were induced to undergo apoptosis by anti-Fas antibody (0.1 μg/ml) for three hours. Subsequently, both control cells (A and C) and apoptotic cells (B and D) were then incubated with 50 μM of DDC in the presence (C and D) and absence (A and B) of 1 μM of rottlerin, an inhibitor of PKCδ. Prior to FACS analysis, cells were also co-stained with propidium iodide (PI), a marker of membrane disintegration during late phases of cell death. In the shown dot plot, the left lower quadrant represents the healthy, non-stained fraction of cells. The right lower quadrant represents the newly formed population of cells in the early stages of apoptosis. These cells are PLS-activated cells, yet still maintaining membrane integrity and thus excluding PI. Cells binding both DDC and PI, i.e., cells in the late stages of apoptosis are represented in the right upper quadrant. The induction of apoptosis was associated with the emergence of a marked, distinct population of cells in the early stages of the apoptotic process, selectively binding DDC and occupying the right lower quadrant of the plot. Inhibition of PLS by rottlerin markedly reduced this cell population (from 46% to 7.9%, B versus D, respectively). UV axis denotes fluorescence intensity of DDC; FL₂ axis denotes fluorescence intensity of PI.

FIG. 4: Jurkat cells were induced to undergo apoptosis by treatment with anti-Fas Ab (0.1 μg/ml) for three hours. Subsequently, cells were co-incubated with 50 μM of DDC and PI, in the presence of 1 mM of CaCl₂, or under conditions of calcium deprivation (i.e., co-administration of the calcium chelators BAPTA and EGTA). Binding of DDC and PI was evaluated by FACS.

FIG. 4A: A and C: control cells; B and D: apoptotic cells; A and B: with Ca²⁺; C and D: deprivation of Ca²⁺. The lower left quadrant in each dot plot represents the healthy non-stained fraction of cells. The lower right quadrant represents the newly formed population of cells in the early stages of apoptosis, characterized by activation of PLS, but still maintaining membrane integrity. As shown, these cells manifested marked DDC binding while excluding PI. However, DDC binding to the cells in early apoptosis was markedly lost upon inhibition of PLS by calcium deprivation (B versus D).

FIG. 4B shows quantification of uptake of DDC by cells in the early stages of apoptosis (solid, dark columns). Uptake was blocked by inhibition of PLS through Ca²⁺ deprivation. As an internal control, binding of DDC to cells in the late stages of the apoptotic process is shown by the dotted columns. Upon losing cell membrane integrity in these cells, molecules can freely enter the cells independently of PLS, and therefore no effect of PLS inhibition by Ca²⁺-deprivation on these cells in the late stages of the apoptotic process was observed.

FIG. 5: Jurkat cells were induced to undergo apoptosis by treatment with anti-Fas Ab (0.1 μg/ml) for three hours, in the absence or presence of the pan-caspase inhibitor Z-VAD-fmk and then were incubated with DDC for 5 min. Shown is the number of events (counts, y-axis) versus fluorescence intensity (UV, x-axis), reflecting binding of DDC to these cells. As demonstrated, induction of apoptosis caused the emergence of a large distinct population of cells in the early stages of apoptosis, manifesting marked DDC binding (dot-dash line, peak B). Treatment with the pan-caspase inhibitor (dashed line) abolished this DDC binding, reflected by return of fluorescence values to the baseline levels of the control, untreated cells (solid line, peak A). Importantly as an internal control, no significant alterations were observed in the binding of DDC to the cells in the late stages of apoptosis (peak C), wherein DDC binding is not a PLS-dependent process, but rather an entry through the disintegrated membrane.

FIG. 6: Control and activated platelets were co-incubated with DDC (at a final concentration of 5 μM under three different conditions: no further treatment; Ca²⁺-deprivation due to incubation with BAPTA and EGTA; and restoration of Ca²⁺ following re-addition of Ca²⁺ and calcium-ionophore A23187. Platelets were then subjected to FACS analysis.

A. FACS histogram; UV axis represents the fluorescence intensity of DDC, while the counts axis represents the number of platelets. While intact platelets (thin solid line) showed low levels of fluorescence intensity upon incubation with DDC, significant shift to higher fluorescence values were manifested by the activated platelets (thick solid line), reflecting marked DDC uptake. Upon Ca²⁺-deprivation, DDC binding was markedly reduced, as reflected by a shift of the platelet population to lower fluorescence values (dotted line). However, DDC binding was completely restored upon restoration of Ca²⁺ (dashed line).

B. Quantification of fluorescence signal, calculated as the geomean of intensity of DDC fluorescence, upon the different treatments of the platelet population. Activated platelets manifested marked enhancement of fluorescence as compared to controls, reflecting selective binding and uptake of DDC upon PLS activation. Inhibition of the PLS system by Ca²⁺ deprivation markedly reduced DDC uptake, and said binding was completely restored upon PLS-reactivation by replenishment of intracellular Ca²⁺. Uptake of DDC by activated platelets is therefore a PLS-dependent process.

FIG. 7 Jurkat cells were induced to undergo apoptosis by anti-Fas Ab treatment as described for FIG. 3 above. Subsequently, cells were incubated with 50 μM of NST830 for 5 minutes, co-stained with PI and subjected to FACS analysis. In order to inhibit PLS in apoptotic cells, two approaches were used:

(i). Ca²⁺-deprivation: apoptotic cells were incubated for 30 min at room temperature in the presence of BAPTA (20 μM) and EGTA (1 mM), and then treated with NST830 and PI as above.

(ii). PLS inhibition by rottlerin: prior to treatment with NST830 and PI, apoptotic cells were incubated for 40 min in the presence of 0.5 μM of rottlerin, and then submitted to FACS analysis.

FIG. 7A is a representative dot plot of the FACS analysis, in which the left lower quadrant represents the healthy, non-stained fraction of cells, the right lower quadrant represents the newly formed population of cells in the early stages of apoptosis, and the upper right quadrant represents cells in the late stages of apoptosis. Whereas control culture (a) is characterized by low percentage of apoptotic cells (4%), triggering of the apoptotic process leads to a large population (64%) of cells in early apoptosis, characterized by NST830 binding (b). Inhibition of PLS by Ca²⁺ depletion (c) or rottlerin (d) reduces this NST830-binding population to 5% and 4%, respectively. Activation and inhibition of the PLS is not associated with alterations in the population of cells in late apoptosis. UV axis denotes fluorescence intensity of NST830; FL₂ axis denotes fluorescence intensity of PI.

FIG. 7B describes quantification of the relative change in the population of early apoptotic cells binding NST830 (light, dotted columns), in comparison to binding of the compound to cells in late apoptosis (solid dark columns), following activation and inhibition of the PLS system. The data are taken from FIG. 7A. The population of cells in late apoptosis cell is not affected by modulation of the PLS system, and the percentage of NST830-binding, PI-permeable cells in both control and anti-Fas treated cultures, regardless of Ca²⁺-deprivation or rottlerin treatment. However, upon induction of apoptosis, there is a marked increase in the percentage of cells that are bind NST830 but exclude PI, i.e., cells in early apoptosis. This increase is both Ca²⁺- and rottlerin-dependent. The Y-axis denotes the percent of positive events.

FIG. 8: Jurkat cells were induced to undergo apoptosis by treatment with staurosporine. Subsequently, these cells were treated with DDC. The relative change in DDC uptake by cells in early versus late stages of apoptosis, following induction and inhibition of the PLS system is presented. Shown are the percents of positive early or late apoptotic events (mean values), calculated from 3 independent experiments, and wherein the events (either early or late) observed in apoptotic culture are regarded as 100%. Selective accumulation of DDC in early, but not in late apoptotic cells was detected as a result of staurosporine treatment. Ca²⁺-deprivation or rottlerin treatment were performed as described in FIG. 1. PLS inhibition by either of these conditions dramatically reduced uptake of DDC into cells in early apoptosis to 14% and 30%, respectably. Importantly, DDC binding to cells in late apoptosis was not effected by modulation of the PLS system.

### EXAMPLES

### Example 1

### Inactivation of PLS Inhibits Transport of DDL into Apoptotic Cells: Ca²⁺-Deprivation Studies

Cultured Jurkat cells (human adult T cell leukemia cells) were grown in suspension in RPMI medium (Beit-Haemek, Israel), supplemented with 10% of fetal calf serum (FCS), 4 mM of L-glutamine, 1 mM of sodium pyruvate, 1 mM CaCl2 and antibiotics (100 units/ml penicilin; 100 μg/ml streptomycin and 12.5 units/ml of nystatin). Prior to induction of apoptosis, medium was replaced with HBS buffer (10 mM HEPES; 140 mM NaCl, 1 mM CaCl). Apoptosis was then triggered by treatment with anti-Fas Ab (0.1 μg/ml; 3 hrs).

Such a procedure culminates in accumulation of a large percentage of apoptotic cells within the culture. Culture was then incubated with DDL at a final concentration of 1 μM for 5 minutes, and then taken immediately for fluorescent microscopy [Olympus fluorescent microscope, model IX70, excitation wavelength of 360 nm, emission at 530 nm].

Selective and fast uptake of DDL by the apoptotic cells, wherein PLS was activated, was detected in the culture, while intact cells not undergoing apoptosis did not bind manifest significant DDL (FIG. 1). DDL fluorescence was observed in the cytoplasm of those cells but not within the nuclei.

Modulation of PLS activity was achieved by the calcium depletion-repletion procedure (Momchilova, A., et al., 2000). Inhibition of PLS by Ca²⁺-deprivation was performed by cell incubation with the extra-and intracellular chelating agents EGTA (Ethylen glycol-bis (β-aminoethyl ether)-N,N,N′-tetraacetic acid, 1 mM) and BAPTA-AM ([1,2-bis(o-Amino-phenoxy) ethane-N,N,N′,N′-tetraacetic acid tetra-(acetoxymethyl) ester, 20 μM, respectively. Such a procedure eliminates any traces of Ca²⁺ possibly present either intra- or extra-cellularly. Subsequent to the induction of apoptosis for three hours, cells were subjected to said PLS inhibition for 30 minutes. Cells were then incubated with DDL for 5 minutes, and subjected to fluorescent microscopy. Inhibition of PLS caused a marked decrease in DDL uptake by the cells, resulting in a marked reduction in fluorescence intensity.

For re-activation of PLS, the EGTA/BAPTA-treated cells were now treated with CaCl (1 mM) and calcium ionophore A23187 (2 μM, Calbiochem, Darmstadt, Germany), thus elevating ambient Ca²+ levels. Upon such PDT activation, the apoptotic cells exhibited a fast, selective and marked uptake of DDL.

The dependence of DDL uptake by the apoptotic cells on PLS activity was also demonstrated by flow cytometric (FACS) analysis. For this purpose, cultured, aged HeLa S3 cells (ATCC CCL-2.2) were grown in Dulbecco's modified Eagle's medium (DMEM), containing CaCl (1.8 mM) and supplemented with 2 mM of L-glutamine; 100 units/ml of penicillin; 100 μg/ml of streptomycin; 12.5 units/ml of nystatin and 10% of fetal calf serum (FCS). Cells were seeded at a density of 5×10⁶ cells/plate on a 10 cm³ culture plates in a volume of 10 ml, and were allowed to age in culture for 96 hours without exchange of the growing medium. Such a procedure culminates in a large percentage of apoptotic cells within the culture. Culture (1×10⁷/ml) was then incubated with DDL at a final concentration of 5 μM in HBS for 5 minutes, and taken immediately for FACS analysis for detection of bound DDL, using Becton-Dickinson cell sorter and CellQuest software. Excitation was at 360 nm and emission was measured at 530 nm.

Three different conditions were examined (FIG. 2): PLS-activation by the apoptotic process, PLS-inhibition; and reactivation of inhibited-PLS. The apoptotic cells (i.e., PLS-activated cells) manifested a distinct shift to higher fluorescence levels, indicating marked, selective DDL binding to these cells. PLS inhibition by Ca²⁺-deprivation almost completely abolished this shift. However, reactivation of PLS by Ca²⁺-re-administration, restored this shift, i.e., restored DDL binding and uptake into these cells.

Therefore, this study demonstrates PLS as a powerful system for the fast transmembrane transport of chemical compounds, into cells in which activation of the PLS transporter takes place, e.g., apoptotic cells.

### Example 2

### Inactivation of PLS Inhibits Transport of DDL into Apoptotic Cells

Cultured Jurkat cells were grown as described in Example 1, and apoptosis was then triggered by treatment with anti-Fas Ab (0.1 μg/ml) for three hours, thus inducing PDT activation in these cells.

In order to inhibit PLS activity in the apoptotic cells, rottlerin was used (Calbiochem, Germany). Low doses of rottlerin have been shown to exert selective inhibition of protein kinase Cδ (PKCδ), which in turn regulates the activity of PLS (Frasch, S C., et al., 2000). Subsequent to induction of apoptosis, cells were incubated for 40 minutes with rottlerin, at a concentration of 1 μM, were then subjected to treatment with DDC at a concentration of 250 μM and immediately taken for FACS analysis (FIG. 3).

A marked decrease (from 46% to 7.9%) in DDC binding to cells in the early stages of apoptosis (right lower quadrant) was observed in the presence of rottlerin, indicating that inactivation of PLS resulted in inhibition of DDC uptake by these cells in early apoptosis. Importantly, the population of cells in the late stages of apoptosis (right upper quadrant) served as an internal control. In cells in late stages of apoptosis, membrane disintegration leads to entry of compounds into the cells, independently of PLS activity. Accordingly, this cell population did not manifest alterations in DDC binding in response to rottlerin, thus further emphasizing the above role of PLS in DDC uptake in the early stages of the apoptotic process.

Inhibition of PLS activity in the apoptotic cells was also achieved by reduction of ambient Ca²⁺ levels. Following induction of apoptosis, both control and apoptotic cells (1×10⁷ cells) were subjected to Ca²⁺-deprivation, by co-treatment with BAPTA (20 μM) and EGTA (1 mM) for 30 minutes. Cells were then treated with DDC (250 μM) and propidium iodide (PI). Binding of DDC to these Ca²⁺-deprived cells and to control, Ca²⁺-treated (1 mM) cells was analyzed by FACS, and the dot plot is shown in FIG. 4A. The lower left quadrant in each dot plot represents the healthy fraction of cells. The lower right quadrant represents the newly-formed population of cells in the early stages of apoptosis, still maintaining membrane integrity but characterized by activation of PLS. As shown, these cells manifest DDC binding while excluding PI. The upper right quadrant represents cells in the late stages of apoptosis, characterized by disintegration of the plasma membrane, thus unselectively permeable to compounds including PI, that is normally excluded from healthy cells.

Upon induction of apoptosis and the associated activation of PLS, cells exhibited a marked increase in the uptake of DDC, from 4.11% to 38.4%. However, upon deprivation of Ca²⁺, uptake of DDC by cells in the early stages of apoptosis decreased from 38.4% to 8.89%, thus being similar to uptake levels of the control cells. Importantly as a control, calcium deprivation did not affect binding of DDC to the cells in the late stages of apoptosis, i.e., cells with permeable membranes, thus further manifesting the dependency of DDC uptake on PLS activity in the cells in early apoptosis (FIG. 4B).

This Example further demonstrates PLS as a drug transport system: modulation of PLS activity by either calcium deprivation or by inhibition of PKCδ with rottlerin, respectively regulated the uptake of DDC into the cells in the early stages of apoptosis.

### Example 3

### Inhibition of PLS by Inactivation of the Apoptotic Machinery Inhibits Uptake of DDC

Induction of apoptosis is a major pathway for the activation of PLS. Therefore, in order to demonstrate the dependency of cellular uptake of PDTCs such as DDC upon PLS activity, modulation of the apoptotic cellular machinery was used as a means for PLS modulation. For this purpose, apoptosis was triggered in cultured Jurkat cells by treatment with anti-Fas antibody. Inhibition of the apoptotic process was performed by the pan-caspase inhibitor Z-VAD-fmk (N-benzyloxycarbonyl-Val-Ala-Asp-fluoromethyl ketone). Uptake of DDC to the control-untreated cells, the apoptotic cells and the pan-caspase-inhibited cells were compared by FACS analysis (FIG. 5).

Jurkat cells (1×10⁶) were treated with anti-Fas Ab (0.1 μg/ml) for 3 hours at 37° C. in the presence and absence of 50 μM of the Z-VAD-fmk inhibitor (Enzyme systems products, USA). Non-treated cells served as controls. Following treatment, cells were centrifuged and incubated with 50 μM of DDC for 5 minutes, and then taken for FACS analysis. FIG. 5 shows the number of events (counts, y-axis) versus fluorescence intensity (UV, x-axis), reflecting binding of DDC to the cells. As shown, induction of apoptosis caused the emergence of a large distinct population of cells in the early stages of apoptosis, manifesting marked DDC binding (dot-dash line, peak B). Treatment with the pan-caspase inhibitor (dashed line) abolished this DDC binding, reflected by return of fluorescence values to the baseline levels of the control, untreated cells (solid line, peak A). Importantly as an internal control, no significant alterations were observed in the binding of DDC to cells in the late stages of apoptosis, wherein DDC binding is not a PLS-dependent process (peak C), thus further emphasizing the dependency of DDC uptake into the cells in early apoptosis on PLS activity.

### Example 4

### Uptake of DDC into Activated Platelets is PLS-Dependent

The selective binding of DDC to activated platelets was determined using flow cytometric (FACS) analysis. Platelet rich plasma was obtained from healthy volunteers. 10⁹ fresh platelets were centrifuged (5 minutes, 380×g), washed and re-suspended in Tyrode's buffer (137 mM NaCl; 2.8 mM KCl; 1 mM MgCl₂, 12 mM NaHCO3; 0.4 mM Na2HPO4; 5.5 mM D-glucose and 10 mM Hepes pH 7.4; 0.35% BSA). Control platelets were kept on ice.

For activation, 200 μl of washed platelets were incubated with the calcium ionophore A23187 in the presence of 5 mM of CaCl₂, at room temperature. Following incubation, platelets were centrifuged (2 min., 10⁴ rpm) and re-suspended in 1 ml of Tyrode's buffer. Activated platelets were subsequently incubated with 5 μM of DDC for 5 minutes at room temperature, under three different conditions: (i). without any further treatment; (ii). under conditions of Ca²⁺-deprivation, induced by 20 μM of BAPTA and 1 mM of EGTA (for 30 min. at 4° C.); (iii). upon restoration of Ca²⁺, wherein platelet intracellular Ca²⁺ levels were replenished by addition of 1 mM of CaCl₂ and 2 μM of the calcium ionophore A23187. Non-activated platelets served as control. Platelets were subjected to flow cytometric (FACS) analysis using Becton-Dickinson cell sorter and CellQuest software. Excitation was at 360 nm and emission was measured at 530 nm. FACS histogram shown in FIG. 6A demonstrates that upon activation, the control platelets population (thin solid line) underwent a marked shift to higher fluorescence levels (thick solid line), reflecting binding and uptake of DDC by the population of activated platelets.

However, upon PLS inhibition by Ca²⁺-deprivation, uptake of DDC was inhibited, reflected by a shift of the platelet population back to low fluorescence values (dotted line). Upon restoration of platelet intracellular Ca²⁺ by re-addition of CaCl₂ to the medium and administration of Ca²⁺-ionophore, thus reactivating PLS, fluorescence returned to the high values observed for the activated platelets before the depletion of Ca²⁺ (dashed line).

FIG. 6B shows the geomean of fluorescence intensity, reflecting DDC binding to the platelets in the above conditions of PLS activity. While control, non-activated platelets wherein PLS is inactive, demonstrated only minimal uptake of DDC, activated platelets wherein PLS is activated, exhibited a marked shift to higher fluorescence intensity, reflecting marked DDC uptake. Said uptake was markedly dependent upon the level of PLS activity: modulation of said activity by modulation of ambient Ca²⁺ levels, inhibited or promoted DDC uptake accordingly. Uptake of DDC by platelets therefore depends upon the level of PLS activity.

### Example 5

### Uptake of NST830 into Apoptotic Cells and Inhibition Studies

Cultured Jurkat cells were induced to undergo apoptosis by anti-Fas antibody (0.1 μg/ml) for 2.5 hours, as described in Example 1. Subsequently, both control and apoptotic cells were incubated with 50 μM of NST830 for 5 minutes, and uptake was measured by FACS. Prior to FACS analysis, cells were also co-stained with PI. The resultant dot plots are shown in FIG. 7A. Induction of apoptosis was associated with the emergence of a large, distinct population of 64% of cells in the early stages of the apoptotic process, selectively binding NST830 and occupying the right lower quadrant of the plot. No substantial change was observed under these conditions in the percentage of cells in late apoptosis, that are unselectively permeable to PI and NST830.

Inhibition of PLS activity in the apoptotic cells was achieved by two approaches:

(i). Modulation of Ca²⁺ levels.

(ii). Treatment with a PLS inhibitor.

(i). Modulation of PLS Activity by the Ca²⁺ Depletion-Repletion Procedure:

At the end of the 2.5 hour apoptosis-induction period, cells (1×10⁷) were treated with 20 μM of BAPTA and 1 mM of EGTA (for 30 min., at room temperature). After 30 minutes of PLS inhibition, cells were stained with NST830 as specified above, and subjected immediately to fluorescent microscopy (FIG. 7A). NST830 uptake was dramatically reduced upon inhibition of PLS by the above calcium deprivation procedure, and a marked decrease in the percentage of cells in early apoptosis (from 64 to 5%) was observed. However, Ca²⁺-deprivation did not affect the percentage of the PI-permeable, late apoptotic cells.

(ii). Inhibition of PLS by Rottlerin:

After the apoptosis-induction period of 2.5 hours, cells were incubated for 40 minutes with rottlerin, at a concentration of 0.5 μM. Cells were then subjected to treatment with NST830 (50 μM) co-stained with PI, and immediately taken for FACS analysis (FIG. 7A). In the presence of rottlerin, a marked decrease (from 64% to 4%) occurred in the percentage of cells in early apoptosis, which manifested NST830 (right lower quadrant), indicating that inhibition of the PLS system resulted in inhibition of the selective uptake of NST830 by the early apoptotic cells. No change in the percentage of population of cells in late apoptosis (upper right quadrant) was observed in the presence of rottlerin.

The relative change in the number of newly-formed apoptotic cells, that showed increased uptake of DDC, in comparison with the number of cells in late apoptosis with permeable membrane, that bound both DDC and PI, is shown quantitatively in FIG. 7B. While binding of DDC to membrane-permeable, late apoptotic cells was unaffected by changes in the intracellular calcium levels, DDC uptake by cells in early apoptosis was markedly a calcium-dependent process. Similar effects were obtained following rottlerin treatment: a marked lo decrease in the newly-formed population of cells in early apoptosis was observed, whereas no change was detected in the percentage of cells in late apoptosis.

### Example 6

### Uptake of DDC into Staurosporine-Treated Cells is a PLS-Dependent Process

The selective uptake of DDC by apoptotic cells is not dependent upon the type of apoptotic trigger. In order to demonstrate this, cells were treated with staurosporine, a well-substantiated trigger of apoptosis. Staurosporine acts in an apoptotic pathway which is largely distinct from that of anti-Fas Ab. Jurkat cells were grown as described in Example 1, and were subsequently treated for 4 hours with 1 μM of staurosporine (S4400; Sigma, USA). After induction of apoptosis, cells were treated with DDC (250 μM) for 5 minutes, and were then co-stained with PI and subjected to FACS analysis. Non-treated cells served as control.

The percent of early and late apoptotic cells in both cultures, showing uptake of DDC is presented in FIG. 8. Following staurosporine treatment, marked specific uptake of DDC into cells in early apoptosis was detected (from 36% in the control cells to 100% in the apoptotic cells). As shown in FIG. 8, this uptake was highly dependent on the level of PLS activity: PLS-inhibition by either Ca²⁺-depletion or rottlerin treatment (said treatments are as described in Example 1) caused a dramatic reduction in DDC-uptake.

1. A method for selectively targeting a medicinally-useful agent into cells in which a phospholipid scramblase (PLS) transport system is activated (PDT-activated cells), wherein said medicinally-useful agent is a PLS-dependent transported compound (PDTC), said method comprising:
administering said agent to said cells, thereby causing selective transport of said agent into said cells.

2. A method according to claim 1, wherein said cells are one or more of apoptotic cells, injured cells or activated cells.

3. A method according to claim 2, wherein said activated cells are selected from activated platelets, activated white blood cells, activated macrophages and inflammatory cells.

4. A method according to claim 1, wherein said agent comprises a marker moiety that can be identified by visual or instrumental means, and wherein said method further comprises detecting said marker moiety in said cells.

5. A method according to claim 4, wherein said marker moiety contains one or more of a fluorescent, radioactive, X-ray imaging, computerized tomography (CT), single photon emmision computerized tomography (SPECT), positron emission tomography (PET) or magnetic resonance imaging (NM) label.

6. A method according to claim 1, wherein said agent has inherent fluorescent properties.

7. A method according to claim 4 for the detection of a disease characterized by occurrence of excessive apoptosis, degenerative disorders, neuro-degenerative disorders, Parkinson's disease, Alzheimer's disease, Huntington chorea, infective disorders, immune-mediated disorders, AIDS, myelodysplastic syndromes, ischemic toxic insults, or tumors.

8. A method according to claim 4 for the detection of a disease characterized by excessive blood clotting, arterial or venous thrombosis, thrombo-embolism, myocardial infarction, cerebral stroke, deep vein thrombosis, disseminated intravascular coagulation (DIC), thrombotic thrombocytopenic purpura (TTP), sickle cell diseases, thalassemia, antiphospholipid antibody syndrome, systemic lupus erythematosis, or unstable atherosclerotic plaque.

9. A method according to claim 4, for the detection of immune-mediated disorders, systemic lupus erythematosus (SLE), rheumatoid arthritis, scleroderma, or other types of connective tissue disorders; thyroiditis; dermatological disorders, pemphigus or erythema nodosum; autoimmune hematological disorders; autoimmune neurological disorders, myasthenia gravis; vasculitis; inflammatory bowel disorders ulcerative colitis; or organ transplant rejection.

10. A method according to claim 4 for use in monitoring the effect of cytotoxic therapy for cancer; monitoring the response to cytoprotective therapy in diseases characterized by excessive apoptosis; or monitoring of graft survival following organ transplantation.

11. A method according to claim 4 for use in tissue culture studies or animal studies of medical conditions associated with PDT activation, or in research fields selected from apoptosis research, research of cell activation, and cell injury.

12. A method according to claims 1, wherein said agent comprises an effector moiety that induces a desired effect on a cell.

13. A method according to claim 12, wherein said desired effect is a therapeutic effect that comprises modulating a biological activity of the cell, protecting cells against damage, reversing a pathological process or modulating a process of cell death.

14. A method according to claim 13, wherein the cells are apoptotic cells, and wherein said effector moiety modulates cell death.

15. A method according to claim 14, wherein said agent is a caspase inhibitor, a modulator of Bcl-2 or an antioxidant.

16. A method according to claim 12, wherein said effector moiety modulates blood coagulation, has an anti-platelet activity, or has a fibrinolytic activity.

17. A method according to claim 13, wherein the cells are cells of the immune system or inflammatory cells, and wherein said effector moiety has an immuno-modulatory or an anti-inflammatory effect.

18. A method according to claim 17, wherein said cells are selected from white blood cells and macrophages.

19. A method according to claim 12, wherein said agent has cytotoxic activity.

20. A method according to claim 12 for treatment of disease manifested by PDT activation, comprising:
administering a therapeutically-effective amount of PDTC to a subject in need of such treatment.

21. A method according to claim 20, for the treatment of a disease selected from: degenerative disorders, neuro-degenerative disorders, Parkinson's disease, Alzheimer's disease, Huntington chorea, infective disorders, immune-mediated disorders, AIDS, myelodysplastic syndromes, ischemic or toxic insults, organ graft rejection after transplantation.

22. A method according to claim 12, for the treatment of a disease, wherein a beneficial effect may be evident by increase of cell death, and wherein the drug is a cytotoxic agent.

23. A method according to claim 22, for the treatment of cancer.

24. A method according to claim 12, wherein the medicinally useful agent is a modulator of blood coagulation.

25. A method according to claim 24, wherein the modulator of blood coagulation is an anticoagulant, an antiplatelet drug or a fibrinolytic agent.

26. A method according to claim 24, for the treatment of a disease selected from: arterial or venous thrombosis, thrombo-embolism, myocardial infarction, cerebral stroke, deep vein thrombosis, disseminated intravascular coagulation (DIC), thrombotic thrombocytopenic purpura (TTP), sickle cell diseases, thalassemia, antiphospholipid antibody syndrome, systemic lupus erythematosis, or atherosclerosis.

27. A method according to claim 12, wherein the medicinally useful agent is a modulator of the activity of macrophages and/or white blood cells.

28. A method according to claim 27, wherein the medicinally useful agent is an immuno-modulatory drug or an anti-inflammatory drug.

29. A method according to claim 27, for the treatment of immune-mediated disorders selected from: systemic lupus erythematosus (SLE), rheumatoid arthritis, scleroderma, or other types of connective tissue disorders; thyroiditis; dermatological disorders such as pemphigus or erythema nodosa; autoimmune hematological disorders; autoimmune neurological disorders such as myasthenia gravis; vasculitis; inflammatory bowel disorders such as ulcerative colitis; or organ transplant rejection.

30. A method according to claim 1, wherein said agent comprises a trapping enhancing moiety (EM).

31. A method according to claim 1, wherein said medicinally-useful agent comprises hydrophilic and hydrophobic moieties.

32. A method according to claim 1, wherein said medicinally-useful agent has an octanol:water partition coefficient of at least 10:1.

33. A method according to claim 1, wherein said hydrophobic moiety is selected among aromatic groups(s), aliphatic group(s), hydrophobic metal chelate(s) comprising metal atom(s), and combinations thereof.

34. A method according to claim 1, wherein said hydrophobic metal chelate(s) comprise(s) technetium.

35. A method according to claim 1, wherein said hydrophilic moiety is charged at physiological conditions.

36. A method according to claim 1, wherein said hydrophilic moiety is in zwitterion form or is a negatively-charged group at physiological conditions.

37. A method according to claim 1, wherein said agent comprises a compound having the formula C, wherein e is selected among 1, 2 and 3 and C is a group having the formula (I): 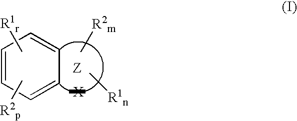
including pharmaceutically acceptable salts and hydrates of the structure of formula (I), wherein said C groups may each be the same or different and;
Z represents a ring system formed of cycloalkyl, cycloalkenyl, heterocyclyl, aryl or heteroaryl groups or combinations of such groups, said ring system consisting of 5-10 atoms;
X represents CH, CH₂, N, NH, O or S;
n, m, r and p are each independently 0 or 1; wherein n+r=1; m+p=1;
R¹ groups may each be the same or different and are independently selected from the group consisting of A, and L-A, wherein L groups may each be the same or different and are independently selected from the group consisting of D, U, U-D, D-U, D-U—O, O—U-D,D-U—NH NH—U-D, D-U-D, and U-D-U;
U stands for a hydrogen or is selected from optionally substituted C₁-C₁₀ C₆ alkylene, C₂-C₁₀ C₆ alkenylene, C₃-C₁₀ C₆ branched alkylene, C₃-C₁₀ C₆ branched alkenylene, C₃-C₆ cycloalkylene, cycloalkenylene, aryl, heterocycloalkylene, heterocycloalkenylene, heteroaryl, and any combinations of said groups;
D is selected from the group consisting of O, S, SO, SO₂, SO₂NH, NHSO₂, NH, PO, PO₂, POOH, PO(NH)₂, NHPOOH, CO, C(O)O, NHCO, CONH, SO₂NHCHCOOH, SO₂NHCO or the corresponding meaning from the above list when D is a bivalent radical;
A groups may each be the same or different and are charged moieties at pH of about 7 when e is 1; or when e is 2 or 3, A groups are independently selected from polar uncharged moieties and charged moieties at pH of about 7, said charged moieties being either positively-charged, negatively-charged or in zwitterion form;
R² is WR³_{b}, where W is null or is selected from the group consisting of secondary or tertiary amine, oxygen, sulfur and D, wherein D is as defined above;
b is 1, 2 or 3,
R³ is selected from the group consisting of hydrogen, C₁-C₆ alkyl, C₂-C₆ alkenyl, C₃-C₈ branched alkyl and C₃-C₈ branched alkenyl and when b is 2 or 3, the R³ substituents may be either the same or different, and
when e is 2 or 3, the C groups are linked to each other either directly or through an L moiety.

38. A method according to claim 37, wherein Cₑ comprises, or is attached through an L group, to a marker moiety or to an effector moiety.

39. A method according to claim 37, wherein said agent comprises a compound of the formula (V): 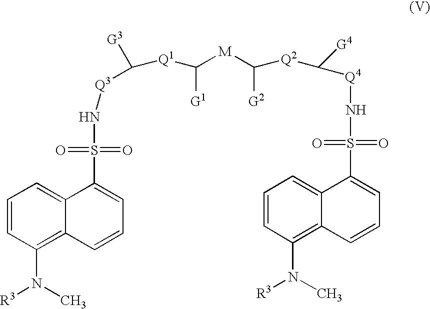
including pharmaceutically acceptable salts and hydrates thereof, wherein G¹, G², G³ and G⁴ groups may be the same or different and are selected independently among hydrogen, COOH, C(O)NH₂, NH₂, and —N⁺(CH₃)₃; M is selected among null, C(O)NH, NH, O, S, S—S, CH₂, (CH₂)₂—, NH(CH₂) N(CH₂)ₖCH(COOH) and N⁺(CH₃)(CH₂)ₖCH(COOH); Q¹, Q², Q³ and Q⁴ groups may be the same or different and are selected among null or (CH₂)ₖ, k being an integer of 1-6, and R³ represents hydrogen or a C₁-C₆ alkyl.

40. A method according to claim 37, wherein said agent comprises a compound of the formula (VIII): 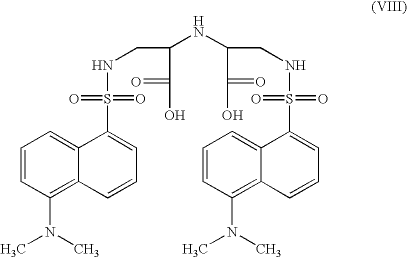
including pharmaceutically acceptable salts and hydrates thereof.

41. A method according to claim 37, wherein said agent comprises a compound of the formula (IX): 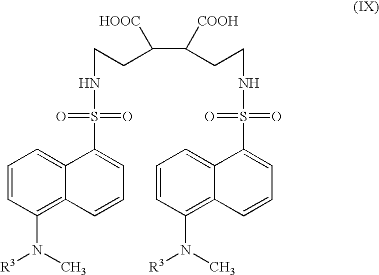
including pharmaceutically acceptable salts and hydrates thereof wherein R³ represents hydrogen or methyl.

42. A method according to claim 37, wherein said agent comprises a compound of the formula (X): 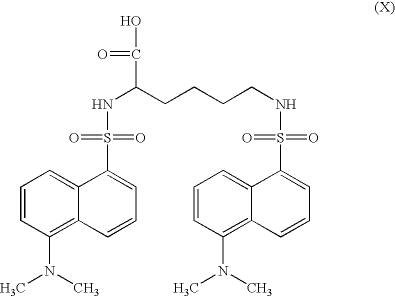
including pharmaceutically acceptable salts and hydrates thereof.

43. A method according to claim 37, wherein said agent comprises a compound of the formula (XI): 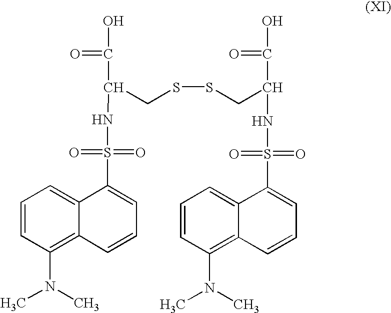
including pharmaceutically acceptable salts and hydrates thereof.

44. A pharmaceutical composition comprising a pharmaceutically acceptable carrier and a medicinally-useful agent capable of being selectively targeted into PDT-activated cells, said medicinally-useful agent being a PDTC.

45. A pharmaceutical composition according to claim 44, wherein said PDT-activated cells are one or more of apoptotic cells, activated cells or injured cells.

46. A pharmaceutical composition according to claim 45, wherein said activated cells are selected from activated platelets, activated cells of the immune system, activated white blood cells and inflammatory cells.

47. A pharmaceutical composition according to claim 44, for detecting apoptotic cells, injured cells or activated cells in a tested subject.

48. A pharmaceutical composition according to claim 44, wherein said agent comprises a marker moiety that can be identified by visual or instrumental means.

49. A pharmaceutical composition according to claim 48, wherein said marker moiety contains one or more of a fluorescent, radioactive, X-ray imaging, CT scan, single photon emmision computerized tomography (SPECT), positron emission tomography (PET) or MRI label.

50. A pharmaceutical composition according to claim 44 wherein said agent has inherent fluorescent properties.

51. A pharmaceutical composition according to claim 48 for the detection of a disease according to claims 7-9.

52. A pharmaceutical composition according to claim 48 for the uses of claims 10-11.

53. A pharmaceutical composition according to claim 44, wherein said agent comprises an effector moiety that induces a desired effect on a target cell.

54. A pharmaceutical composition according to claim 53, wherein said desired effect is a therapeutic effect that comprises modulating a biological activity of the cell, protecting cells against damage, reversing a pathological process or modulating enhancing a process of cell death.

55. A pharmaceutical composition according to claim 54 wherein the target cells are apoptotic cells, and wherein said effector moiety modulates cell death.

56. A pharmaceutical composition according to claim 55 wherein said agent is a caspase inhibitor, a modulator of Bcl-2 or an antioxidant.

57. A pharmaceutical composition according to claim 53, wherein the target cells are activated platelets or apoptotic cells, and wherein said effector moiety inhibits blood coagulation, has an anti-platelet activity, or has a fibrinolytic activity.

58. A pharmaceutical composition according to claim 53, wherein the target cells are cells of the immune system or inflammatory cells, and wherein said effector moiety has an immuno-modulatory effect or an anti-inflammatory effect.

59. A pharmaceutical composition according to claim 58, wherein said inflammatory cells are selected from white blood cells and macrophages.

60. A pharmaceutical composition according to claim 53, wherein said agent has cytotoxic activity.

61. A pharmaceutical composition according to claim 53 for the treatment of a disease according to any of claims 21 to 29.

62. A pharmaceutical composition according to claim 44, wherein said agent comprises a TEM.

63. A pharmaceutical composition according to claim 44, wherein said medicinally-useful agent comprises hydrophilic and hydrophobic moieties.

64. A pharmaceutical composition according to claim 44, wherein said medicinally-useful agent has an octanol : water partition coefficient of at least 10:1.

65. A pharmaceutical composition according to claim 63, wherein said hydrophobic moiety is selected among aromatic groups(s), aliphatic group(s), hydrophobic metal chelate(s) comprising metal atom(s), and combinations thereof.

66. A pharmaceutical composition according to claim 63, wherein said hydrophobic metal chelate(s) comprise(s) technetium.

67. A pharmaceutical composition according to claim 63, wherein said hydrophilic moiety is charged at physiological conditions.

68. A pharmaceutical composition according to claims 63, wherein said hydrophilic moiety is in zwitterion form or is a negatively-charged group at physiological conditions.

69. A pharmaceutical composition according to claim 44, wherein said agent comprises a compound of formula (I) as defined in claim 37, or a conjugate of the compound of formula (I) with a marker moiety or an effector moiety, as defined in claim 37.

70. A method for screening for and identifying compounds to be selectively delivered to PDT-activated cells (PDTC), comprising choosing from candidate compounds those compounds manifesting transport into PDT-activated cells in a PLS-dependent manner.

71. A method according to claim 70 comprising:
(a) providing a candidate compound and a cellular system, wherein PLS and PKCδ are expressed and functional;
(b) providing conditions suitable for activation of PDT;
(c) placing known concentrations of the candidate compound outside the cell, and measuring the concentration of said compound within the cell (i.e., the intracellular concentration) after a predetermined time period; and
(d) repeating steps (a) and (c), but replacing step (b) by providing conditions under which PDT activity is inhibited;
a compound wherein a ratio of >2 is measured between the intracellular concentration achieved upon activation of PDT, and the intracellular concentration achieved upon inhibition of PDT, being a likely candidate for a PDTC.

72. Use of a medicinally-useful agent, being a PDTC, that can be transported into PDT-activated cells by PLS, for the preparation of a pharmaceutical composition for selectively targeting said agent into said cells.

73. Use according to claim 72, wherein said cells are one or more of apoptotic cells, activated cells or injured cells.

74. Use according to claim 72, for the manufacture of a pharmaceutical composition according to any one of claims 44 to 69.

75. Use according to claim 72, wherein said agent comprises a compound of the formula (I) as defined in claim 37.

76. Use according to claim 71, wherein said agent comprises a compound as defined in any one of claims 36 to 43.
